(19) United States
(12) Patent Application Publication
(10) Pub. No.: US 2021/0069254 A1
(43) Pub. Date: Mar. 11, 2021

(54) COMPOSITIONS FOR EXTRACELLULAR VESICLE STORAGE AND FORMULATION

(71) Applicant: Evox Therapeutics Ltd, Oxford (GB)

(72) Inventors: André GÖRGENS, Huddinge (SE); Samir EL ANDALOUSSI, Huddinge (SE); Oscar WIKLANDER, Solna (SE); Giulia CORSO, Stockholm (SE)

(21) Appl. No.: 16/963,883

(22) PCT Filed: Feb. 11, 2019
(86) PCT No.: PCT/EP2019/053312

§ 371 Date: Jul. 22, 2020

(30) Foreign Application Priority Data
Feb. 9, 2018 (GB) 1802163.4

(51) Int. Cl. A61K 35/28; A61K 47/18; A61K 47/02; A61K 47/26; A61K 47/42

(52) U.S. Cl. CPC A61K 35/28; A61K 47/183; A61K 47/42; A61K 47/26; A61K 47/02

(57) ABSTRACT

The present invention relates to compositions and buffers for storing engineered extracellular vesicles (EVs), such as exosomes, for extended time periods with maintained stability and function. The present invention allows for both optimized long-term storage and for clinical application of the stored EVs.

### TECHNICAL FIELD

The present invention relates to compositions and buffers for storing (e.g. genetically engineered) extracellular vesicles (EVs), such as exosomes, for extended time periods with maintained stability and function.

### BACKGROUND ART

Extracellular vesicles (EVs), such as exosomes, are nano-sized lipid vesicles with tantalizing utility for drug delivery and as drug modalities in their own right. EVs have major manufacturing and toxicology advantages over cell therapies such as the ability to increase sterility assurance in the process using microbial sterility filters, but EVs are also non-replicating which lowers the risks for adverse tumorigenic and immunogenic responses, unlike the highly immune-stimulatory potential of gene therapy vectors such as AAVs and lentiviruses. EVs are also more amenable than other advanced therapies to chemistry, manufacturing and control (CMC) considerations, such as drug storage temperature optionality. However, optimal conditions, buffers, and methods for long-term storage of EVs with maintained stability and activity has not been found. The academic literature contains discrete but incomplete descriptions of different storage conditions, but neither of these have been developed or adapted for modified EVs. For instance, Bosch et al. Sci Reports, 2016, has in an initial attempt shown that trehalose can improve stability of primary cell derived exosomes when stored in PBS. Although the addition of trehalose appears to have been beneficial for native, unmodified EVs the publication in question leaves considerable room for improvement, especially as it does not address the multifactorial nature of EVs, and in particular engineered EVs, and does not address the many distinct challenges associated with storage of EVs, i.e. not only maintained particle numbers but also maintained integrity, bioactivity, surface properties, and stability of externally facing and luminal drug cargoes such as protein biologics or RNA therapeutics. For instance, the very recent patent application WO2018/070939 discusses buffers for enhancing the stability during lyophilization of native, un-engineered mesenchymal stromal cell (MSC)-derived exosomes. Similarly, in U.S. Pat. No. 6,812,023, native exosomes from dendritic cells (Dexosomes) or from tumor cells (Texosomes) are formulated in a buffer which intends to enhance vesicle stability, more specifically retaining the holistic inherent biological activity of unmodified exosomes.

EVs such as exosomes are typically produced in a few simple steps: (1) culturing of a suitable EV-producing cell source, which may be, e.g., a cell line or primary cells, in a suitable culture vessel, (2) harvesting of the supernatant, i.e. the cell culture medium into which EVs are secreted by the EV-producing cells, and (3) purification (also known as isolation) of the EVs to a suitable level of purity depending on the application in question. However, reproducible methods for storing EVs are lacking in the art, and in particular methods and compositions for storing engineered, non-native EVs which have a particular therapeutic and/or pharmacological activity. Furthermore, methods and compositions for formulating non-native EVs for clinical use are also absent, and the current state-of-the-art typically merely uses phosphate-buffered saline as the vehicle of choice for both storage and drug formulation of EVs for in vivo use.

### SUMMARY OF THE INVENTION

The present invention aims to address the problems of the current art and to overcome the challenges associated with devising an optimal buffer composition which may be used both for long-term storage of EVs, such as genetically engineered EVs, but also for clinical application of such modified EVs. The present invention achieves these and other objectives by utilizing a judicious combination of solutions and agents which minimize aggregation of EVs, maximize the stability and integrity of individual EVs and EV populations, and enables maintaining stable physico-chemical conditions across a wide range of temperatures and conditions. Importantly, and in complete contrast to the prior art, the present invention enables long-term storage of engineered EVs, specifically EVs that have been genetically modified to comprise either exogenous and/or endogenous drug cargoes, e.g. mRNA, short RNAs such as siRNA and antisense oligonucleotides, polypeptide-based therapeutics such as antibodies, receptors, enzymes and/or peptides, as well as other drug cargoes.

Key examples of genetically engineered EVs include EVs that have been genetically engineered to comprise at least one fusion protein between an exosomal protein and a therapeutic protein of interest. Such therapeutic proteins of interest are often referred to herein as drug cargoes or drugs and may be selected from a wide variety of proteins or peptides with therapeutic activity or the ability to target tissues or organs or cells of interest. Non-limiting examples of such protein-based drug cargoes include, transmembrane proteins, enzymes or transporters implicated in lysosomal storage disorders, for instance NPC1, cystinosin, CLN3, CLN6, GBA, AGAL, etc., but they may also include antibodies or antibody derivatives (single-domain antibodies, single-chain fragments, etc.) or receptors, cytokines, or peptides. Other types of genetically engineered EVs may include EVs which have been endogenously loaded via genetic engineering of drug cargo which include messenger RNA and short hairpin RNA or microRNA. All of these nucleic acid based drug modalities can be genetically loaded into the engineered EVs with the help of RNA-binding proteins fused to an exosomal protein, whereby the RNA cargo molecule is transported into the EV in connection with the formation of the EV in the EV-producing cell.

In a first aspect, the present invention relates to storage buffers for storing EVs, in particular genetically engineered EVs, for extended periods of time. The storage buffer typically comprises (i) an aqueous solution comprising at least one buffering agent, (ii) at least one stabilizing component comprising a saccharide moiety, and (iii) at least one polypeptide.

In another aspect, the present invention relates to EV compositions which comprise the storage buffers herein and at least one population of EVs. Such EV compositions may thus comprise (i) an aqueous solution comprising at least one buffering agent, (ii) at least one component comprising a saccharide moiety, (iii) at least one exogenously added polypeptide, and (iv) importantly at least one population of EVs, such as genetically engineered EVs.

In yet another aspect, the present invention relates to a pharmaceutical composition comprising the EV compositions as described herein and a pharmaceutically acceptable carrier. Such pharmaceutical compositions may be used in medicine, and specifically in the prophylaxis, treatment, and/or alleviation of various diseases.

Surprisingly, the present inventors have found that the buffers herein maintain the activity of luminal, transmembrane, and extravesicular EV drug cargoes. This realization is highly unexpected, as the presence of an exogenously added polypeptide (for instance human serum albumin) to the storage and formulation buffer results has been surmised to result in the formation of a corona around the EVs, which has been considered a hindrance for EV-mediated delivery, drug release and/or activity of transmembrane and/or extravesicular drug cargo. Importantly, the present inventors have devised inventive methods of preparing such novel stability-enhancing buffers, by utilizing particular concentrations of the key buffer components and by using inventive methods of preparing the buffers herein.

Thus, in a further aspect, the present invention relates to a method for preparing the pharmaceutical compositions herein. Such methods typically comprise a one-step mixing of an EV composition with a pharmaceutically acceptable carrier, as above-mentioned. The present invention also pertains to methods and processes for producing the EV compositions as described herein. Such processes may comprise the steps of (i) culturing of EV-producing cells in a suitable culture vessel, (ii) purify and/or isolate (partly or completely, depending on the level of desired purity of the final EV composition) the EVs obtainable from the EV-producing cells, and (iii) at any point prior to, during, and/or after step (ii) mixing the EVs with the storage buffer herein.

In yet another aspect, the present invention relates to a method for stably storing EVs, comprising the steps of (i) introducing EVs into the storage buffers as described herein, and (ii) storing the EV-containing storage buffer at a suitable temperature. Suitable temperatures for storage include sub-zero temperatures, although temperatures above 0° C. are also contemplated and work well for short- and medium-term storage of EVs with maintained activity.

Thus, the present invention provides for novel methods, compositions, pharmaceutical compositions and associated process for storing EVs such as exosomes for extended time periods with maintained stability, integrity, and without any negative effects on EV function and/or activity. This is key to successful clinical use of EV therapeutics and the present invention is particularly important for drug loaded and/or engineered EVs and exosomes and enables seamless clinical application of EVs from conventional upstream and downstream processes.

### BRIEF DESCRIPTION OF THE FIGURES

FIG. 1. EV concentration stability. i) Nanoparticle tracking analysis (NTA) of Mesenchymal stromal cell (MSC) derived EVs obtained from a genetically modified immortalized MSC cell line analyzed direct following isolation (fresh) or stored in different buffers (A-I) and different temperatures (−80° C., −20° C., 4° C.) for 26 weeks. li) Green fluorescent protein (GFP)-positive EVs derived from genetically modified HEK293T stable cells (HEK) analyzed by spectrometer (SpectraMax) after 26 weeks storage in different buffers (A-I) and different temperatures (−80° C., −20° C., 4° C.) for 26 weeks. Yellow bars indicate the best conditions at −80° C.

FIG. 2. Stability of EV function. i) Assessment of cellular uptake in Huh7-cells of GFP-positive genetically engineered EVs derived from stably modified HEK cells analyzed by flow cytometry after 26 weeks storage in different buffers (A-I) and different temperatures (−80° C., −20° C., 4° C.) for 26 weeks. ii) NF-κB reporter (Luc)-HEK293 cells genetically modified to express the NF-κB-luciferase reporter gene, was employed to assesses the anti-inflammatory capacity of MSC derived EVs which are endogenously loaded with TNFR by the genetically modified cells to display TNFalpha decoying receptors. The level of inflammation was assessed by luciferase activity in the unstimulated cells (cells), TNFalpha stimulated cells (TNFalpha) and TNFalpha stimulated cells with PBS (PBS) or EVs stored in different buffers (A-I) and different temperatures (−80° C., −20° C., 4° C.) for 26 weeks. Yellow bars indicates the best conditions at −80° C.

### DETAILED DESCRIPTION OF THE INVENTION

The present invention relates to compositions and buffers for storing (e.g. genetically engineered) extracellular vesicles (EVs), such as exosomes, for extended time periods with maintained stability and activity.

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the exogenous polypeptides described in connection with the storage buffers are to be understood to be disclosed, relevant, and compatible with all other aspects, teachings and embodiments herein, for instance aspects and/or embodiments relating to the pharmaceutical compositions herein. Furthermore, certain embodiments described in connection with certain aspects, for instance saccharide and/or saccharide moieties described in connection with the storage buffer are to be understood to be included, relevant, applicable, and compatible with the EV-containing composition and pharmaceutical formulations herein.

The terms “extracellular vesicle” or “EV” are used interchangeably herein and shall be understood to relate to any type of vesicle that is obtainable from a cell in any form, for instance an exosome (e.g. any vesicle derived from the endo-lysosomal pathway), a microvesicle (e.g. any vesicle shed from the plasma membrane of a cell), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. The sizes of EVs may vary considerably but an EV typically has a nano-sized hydrodynamic radius, i.e. a radius below 1000 nm. Clearly, EVs may be derived from any cell type, both in vivo, ex vivo, and in vitro. Furthermore, the said terms shall also be understood to relate to extracellular vesicle mimics, cell membrane-based vesicles obtained through for instance membrane extrusion, sonication, or other techniques, etc. Importantly, exosomes represent the most preferred type of EV and unless otherwise indicated exosomes are suitable for and applicable to all teachings, alternatives, and applications of EVs herein. It will be clear to the skilled artisan that when describing medical and scientific uses and applications of the EVs, the present invention normally relates to a plurality of EVs, i.e. a population of EVs which may comprise thousands, millions, billions or even trillions of EVs. EVs may be present in various concentration ranges and concentrations such as for instance 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml), or any other number larger, smaller or anywhere in between. In the same vein, the term “population” shall be understood to encompass a plurality of entities which together constitute a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, drug cargo comprised in the EVs, any targeting moieties present on the EVs, the characteristics of the pharmaceutical formulation, etc. As above-mentioned, the EVs of the present invention may comprise various types of therapeutic agents (drug cargoes), i.e. the EVs are genetically engineered to comprise a particular drug cargo such as an mRNA, an shRNA, a miRNA, a protein, a peptide, etc., or the EVs are genetically engineered to enhance their activity as drug delivery vectors for exogenous drug cargoes, for instance small molecule drugs or chemically synthesized protein-based and/or RNA-based drugs As abovementioned, drug cargoes may be genetically engineered into the genetically modified drug-loaded EVs with the aid of exosomal polypeptides fused to a particular therapeutic protein or interest or to any other protein which may enable loading of another, separate drug cargo, for instance an RNA therapeutic (such as an mRNA, an shRNA, an siRNA, etc.) or a small molecule drug cargo. Such drug cargoes may thus be at least one therapeutic small molecule drug. In some embodiments, the therapeutic small molecule drug may be selected from the group consisting of DNA damaging agents, agents that inhibit DNA synthesis, microtubule and tubulin binding agents, anti-metabolites, inducers of oxidative damage, anti-angiogenics, endocrine therapies, anti-estrogens, immuno-modulators such as Toll-like receptor agonists or antagonists, histone deacetylase inhibitors, inhibitors of signal transduction such as inhibitors of kinases, inhibitors of heat shock proteins, retinoids, inhibitors of growth factor receptors, anti-mitotic compounds, anti-inflammatories, cell cycle regulators, transcription factor inhibitors, and apoptosis inducers, and any combination thereof. In further embodiments, the drug cargo may be a therapeutic nucleic acid-based agent. Such nucleic acid-based therapeutic agents as abovementioned may be selected from the group comprising single-stranded RNA or DNA, double-stranded RNA or DNA, oligonucleotides such as siRNA, splice-switching RNA, CRISPR guide strands, short hairpin RNA (shRNA), miRNA, antisense oligonucleotides, polynucleotides such as mRNA, plasmids, or any other RNA or DNA vector. Of particular interest are nucleic acid-based agents which are chemically synthesized and/or which comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc. In yet further embodiments, the EVs as per the present invention may comprise drug cargoes which may be protein and/or peptides. Such proteins and/or peptides which are engineered to be loaded as drug cargoes into the EVs may be present inside of the EVs, inserted into the EV membrane (for example in the case of transporter proteins) or in association with the EV membrane (for instance in a transmembrane fashion), or may be protruding from the EV into the extravesicular environment. Such therapeutic protein and/or peptide agents, i.e. drug cargoes, may be selected from a group of non-limiting examples including: antibodies, intrabodies, single chain variable fragments (scFv), affibodies, bi- and multispecific antibodies or binders, affibodies, darpins, receptors, ligands, enzymes for e.g. enzyme replacement therapy or gene editing, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins, mitochondrial proteins, and heat shock proteins, etc. In a preferred embodiment, the drug cargo may be a CRISPR-associated (Cas) polypeptide (such as Cas9) with intact nuclease activity which is associated with (i.e. carries with it) an RNA strand that enables the Cas polypeptide to carry out its nuclease activity in a target cell once delivered by the EV. Alternatively, in another preferred embodiment, the Cas polypeptide may be catalytically inactive, to enable targeted genetic engineering. Yet another alternative may be any other type of CRISPR effector such as the single RNA-guided endonuclease Cpf1 (from species such as Acidaminococcus or Lachnospiraceae). Additional preferred drug cargoes include therapeutic proteins selected from the group comprising enzymes for lysosomal storage disorders, for instance glucocerebrosidases such as imiglucerase, alpha-galactosidase, alpha-L-iduronidase, iduronate-2-sulfatase and idursulfase, arylsulfatase, galsulfase, acid-alpha glucosidase, sphingomyelinase, galactocerebrosidase, galactosylceramidase, glucosylceramidase ceramidase, alpha-N-acetylgalactosami nidase, beta-galactosidase, lysosomal acid lipase, acid sphingomyelinase, NPC1, NPC2, heparan sulfamidase, N-acetylglucosaminidase, heparan-a-glucosaminide-N-acetyltransferase, N-acetylglucosamine 6-sulfatase, galactose-6-sulfate sulfatase, galactose-6-sulfate sulfatase, hyaluronidase, alpha-N-acetyl neuraminidase, GlcNAc phosphotransferase, mucolipin1, palmitoyl-protein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase. In other preferred embodiments, the therapeutic protein may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as dystrophin, mini dystrophin, utrophin, titin, nebulin, dystrophin-associated proteins such as dystrobrevin, syntrophin, syncoilin, desmin, sarcoglycan, dystroglycan, sarcospan, agrin, and/or fukutin.

The terms “EV enrichment polypeptide”, “EV protein”, “EV polypeptide”, “exosomal polypeptide” and “exosomal protein” and similar terms are used interchangeably herein and shall be understood to relate to any polypeptide that can be utilized to transport a polypeptide construct (which typically comprises, in addition to the exosomal protein, a drug cargo or a protein intended to transport a drug cargo) to a suitable vesicular structure, i.e. to a suitable EV. More specifically, these terms shall be understood as comprising any polypeptide that enables transporting, trafficking or shuttling of a fusion protein construct and/or additional protein and/or nucleic acid-based components to a vesicular structure, such as an EV. Non-limiting examples of such exosomal polypeptides are for instance CD9, CD53, CD63, CD81, CD54, CD50, FLOT1, FLOT2, CD49d, CD71 (also known as the transferrin receptor) and its endosomal sorting domain, i.e. the transferrin receptor endosomal sorting domain, CD133, CD138 (syndecan-1), CD235a, ALIX, syntenin (also known as syntenin-1), the N terminal portion of syntenin, Lamp2b, syndecan-2, syndecan-3, syndecan-4, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, TSG101, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, ARRDC1, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, other exosomal polypeptides, and any combinations thereof, but numerous other polypeptides capable of transporting a therapeutic and/or targeting agent (for instance, a protein, an RNA molecule, or a peptide) within the scope of the present invention. Typically, in many embodiments of the present invention, at least one exosomal polypeptide is comprised a polypeptide construct which further comprises a therapeutic protein of interest, and this fusion polypeptide construct may advantageously also comprise various other components, including linkers, transmembrane domains, cytosolic domains, multimerization domains, release domains, etc.

The terms “source cell” or “EV source cell” or “parental cell” or “cell source” or “EV-producing cell” or any other similar terminology shall be understood to relate to any type of cell that is capable of producing EVs, e.g. exosomes, normally under suitable cell culturing conditions. Such conditions may be suspension cell culture or adherent cell culture or any in other type of culturing system. Hollow-fiber bioreactors and other types of bioreactors represent highly suitable cell culturing infrastructure and so does various bioreactors for suspension cells. The source cells per the present invention may be selected from a wide range of cells and cell lines, for instance mesenchymal stem or stromal cells or fibroblasts (MSCs and fibroblasts are obtainable from e.g. bone marrow, adipose tissue, Wharton's jelly, perinatal tissue, tooth buds, umbilical cord blood, skin tissue, amnion, etc.), amnion cells and more specifically amnion epithelial cells optionally expressing various early markers, etc. Cell lines of particular interest include human umbilical cord endothelial cells (HUVECs), human embryonic kidney (HEK) cells, human amnion epithelial cells, endothelial cell lines such as microvascular or lymphatic endothelial cells, chondrocytes, MSCs, airway or alveolar epithelial cells, and various other non-limiting examples of cell sources. Importantly, the present invention relates in preferred embodiments to cells that have been genetically modified (interchangeably known as “genetically engineered”) to contain at least one polynucleotide of interest, which results in production of genetically engineered EVs comprising a particular biomolecule transcribed from and/or loaded into the EV with the help of the product of the polynucleotide. Unlike in the prior art, a given EV population of the present invention is highly homogenous in terms of the EVs it comprises, i.e. the EVs making up an EV population have very little to no variability between them in terms of endogenous and/or exogenous drug loading and in terms of their surface RNA and protein profile.

In a first aspect, the storage buffers of the present invention comprise (i) an aqueous solution comprising at least one buffering agent, (ii) at least one stabilizing component comprising a saccharide moiety, and (iii) at least one polypeptide. The polypeptide component is typically added exogenously. Importantly, the aqueous solutions which, typically form the bulk of the volume of the storage buffer, are typically isotonic solutions, to enable in vitro, ex vivo and in vivo use of the storage buffers, and more specifically the genetically engineered EVs stored in the storage buffer.

In one embodiment, the aqueous solution may be selected from various solutions comprising suitable ingredients such as salts, sugars, etc. Suitable aqueous solutions may be selected from the group comprising phosphate-buffered saline (PBS), Dulbecco's PBS, Hank's buffered salt solution (HBSS), Earle's balanced salt solution (EBSS), Modified Eagle's Medium, Minimum Essential Medium, Dulbecco's Modified Eagle's Medium (DMEM), Glasgow Minimum Essential Medium (GMEM), RPM11640, IMDM, Ham F10, Ham's F12, Ringer's acetate, Ringer's lactate, Plasmalyte A, and/or any combination thereof.

In a further embodiment, the at least one buffering agent of the storage buffer may be selected from the group comprising MES, bis-tris methane, ADA, ACES, bis-tri propane, PIPES, MOPSO, cholamine chloride, MOPS, BES, TES, HEPES, DIPSO, MOBS, acetamidoglycine, TAPSO, TEA, POPSO, HEPPSO, EPS, HEPPS, tricine, tris, glycinamide, glycylglycine, HEPBS, bicine, TAPS, AMPB, CHES, AMP, AMPSO, CAPSO, CAPS, CABS, and/or any combination thereof. Naturally, various buffering agents may be included alone or in combination. In preferred embodiments, the buffering agent is selected from HEPES and normally sodium salts thereof, HEPPSO and normally HEPPSO hydrate, tris, PIPES, HEPBS, CABS, etc. Suitable concentrations vary for these compounds but generally the optimal range is 1-100 mM, although but lower and higher concentrations and various subranges may be useful.

The storage buffers per the present invention also comprises at least one component which comprises at least one saccharide and/or saccharide moiety. Without wishing to be bound by any theory, it is surmised that the at least one at least one component which comprises at least one saccharide and/or saccharide moiety has a stabilizing function which is important for the short and long-term stability of the EVs comprising in the storage buffer. This is particularly important for engineered exosomes, which may comprise various drug modalities with specific activity which need to be maintained during storage. The component comprising the saccharide and/or saccharide moiety may be a monosaccharide, a disaccharide, a trisaccharide, an oligosaccharide, a polysaccharide, a sugar alcohol, and/or any combination thereof. In a preferred embodiment, the at least one component which comprises at least one saccharide and/or saccharide moiety is a non-reducing disaccharide such as sucrose or trehalose. In alternative embodiments, reducing sugars may also be utilized, for instance lactose and maltose, although trehalose and sucrose appear to result in enhanced EV stability. The concentration of such saccharides and/or saccharide-containing moieties may be in the range of 1-200 mM, preferably 1-100 mM.

Importantly, the inventors behind the present invention has discovered that the addition of at least one exogenously polypeptide is crucial to avoid aggregation of EVs during storage. This is especially the case for sub-zero (Celsius) temperatures but is also important when EVs are stored in supra-zero temperatures. The inventive storage buffers as per the present invention thus comprises at least one exogenous polypeptide, which may be selected from the following group of polypeptide agents: serum albumin, human serum albumin (HSA), recombinant serum albumin, recombinant HSA, Albunorm®, individual amino acids, di-amino acids, tri-amino acids, oligo-amino acids, peptides, and various other polypeptides and combinations thereof.

Concentration ranges may vary significant depending on the concentration and type of EV preparation. For instance, the concentration of e.g. HSA may be anywhere in the range of 50 μg/mL to 5000 μg/mL, so concentrations within this range may be useful but so may higher concentrations, such as e.g. approximately 10000 μg/mL. The fact that at least one exogenously added polypeptide can provide stability enhancement while at the same time retaining in vivo PK/PD profiles of EV compositions is highly surprising, especially for EVs that have been genetically engineered to comprise membrane-associated, transmembrane, and/or extravesicular drug cargoes. Thus, in a preferred embodiment, the present invention relates to genetically engineered EVs comprising a transmembrane and/or extravesicular drug cargo, wherein such EVs are formulated for long-term storage and for in vivo administration in the inventive storage buffers disclosed herein. Thus, in a preferred embodiment, the present invention relates to a storage buffer comprising (i) an aqueous solution comprising at least one buffering agent, (ii) at least one component comprising a saccharide moiety, (iii) least one exogenously added polypeptide, and (iv) genetically modified extracellular vesicles (EVs) comprising an exogenous and/or endogenous drug cargo. In highly preferred embodiments, as abovementioned, such EVs may comprise a membrane-associated, transmembrane, and/or extravesicular drug cargo, for instance a transmembrane protein and/or an extravesicular protein for therapy and/or targeting.

In yet additional embodiments, the storage buffer may further comprise at least one excipient. Such excipient(s) may be selected from a cryoprotectant, a lyophilization excipient, a preservative, an antibiotic, an organic solvent, an inorganic salt (such as ammonium nitrate, calcium chloride, potassium hydrate, etc.), a carbohydrate, an amino acid, a vitamin, a fatty acid, a serum component, a trace element, and/or any combination thereof. One preferred embodiment is DMSO, which may have various functions, e.g. as a cryoprotectant when the storage buffer is stored at sub-zero temperatures. DMSO may be present at concentrations ranging from 0.1% to 10%, preferably 1-5%. Other excipients of relevance may include PEG (polyethylene glycol), heparin, propylene glycol, ethylene glycol, EDTA, glycerol, dithiothreitol (DTT), co-block polymers, and/or any combination thereof.

As above-mentioned, the storage buffers as per the present invention are highly suitable for EVs comprising either an exogenous and/or endogenous drug cargo. Examples of such exogenous drug cargoes include small molecule drugs (typically organic molecules with pharmacological activity), peptides or proteins such as antibodies which may be loaded into and/or onto the EVs, RNA therapeutics such as short interfering RNAs (siRNAs), short hairpin RNAs (shRNAs), messenger RNA, splice-switching oligonucleotides, antisense oligonucleotides, CRISPR-Cas9, and the like. Examples of endogenous drug cargoes (i.e. drug cargos that are loaded into the EVs through engineering and/or any other modification of EV-producing cells) include peptides, proteins, antibodies, mRNAs, shRNAs, miRNAs, CRISPR-Cas9 and any other drug modality.

In another aspect, the present invention relates to EV compositions which comprise the storage buffer as described herein, in combination with EVs, such as genetically engineered EVs. Such engineered EV compositions may thus comprise (i) an aqueous solution comprising at least one buffering agent, (ii) at least one component comprising a saccharide moiety, (iii) at least one exogenously added polypeptide, and (iv) importantly at least one population of EVs. In preferred embodiments, as abovementioned, the EVs are not in their native, unmodified state but they are genetically engineered to comprise at least one drug cargo. The drug cargo may be present inside the EV (intra-vesicular or luminal), in association with the EV membrane or on the external side of the EV membrane (extravesicular). The cargo protein may preferably be a transmembrane protein, transporter protein, or fusion protein comprising a fusion of a therapeutic protein of interest to an exosomal protein. As above-mentioned, the individual EVs which together constitute the EV population may comprise at least one drug cargo or a mix of drug cargoes of one or more type (e.g. one single EV may comprise at least one RNA drug cargo and one protein-based drug cargo, or at least one protein-based drug cargo such as an antibody attached to the outside of the EV combined with a small molecule drug cargo or an RNA cargo comprised essentially inside the EV). Naturally, all the components mentioned above in connection with the storage buffer may be freely included and combined also in the context of the EV composition.

In yet another aspect, the present invention relates to a pharmaceutical composition comprising the EV compositions as described herein and a pharmaceutically acceptable carrier. In preferred embodiments, the pharmaceutical compositions comprise EVs which have been genetically engineered to comprise a drug cargo formulated into the buffers herein and the pharmaceutically acceptable carrier. Such pharmaceutical compositions may be used in medicine, and specifically in the prophylaxis, treatment, and/or alleviation of various diseases, including the following non-limiting examples of diseases, disorders and conditions: Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, Duchenne muscular dystrophy and other muscular dystrophies, lysosomal storage diseases such as Gaucher disease, Fabry's disease, MPS I, II (Hunter syndrome), and III, Niemann-Pick disease type A, Niemann-Pick disease type B, Niemann-Pick disease type C, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sezary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor. In preferred embodiments, the pharmaceutical compositions of the present invention are for use in the treatment of lysosomal storage disorders (LSDs), for instance Alpha-mannosidosis, Beta-mannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV—Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease, as well as other LSDs. The present inventors have realized that there is a surprising disease-modifying synergy between the engineered EVs of the present invention and some of the buffer components described herein, specifically trehalose and sucrose. Trehalose and/or sucrose is believed to upregulate autophagy. Treatment with trehalose is therefore believed to be beneficial in the treatment of diseases resulting from dysregulation of autophagy or diseases which benefit from increased autophagy. Such diseases include many neurodegenerative diseases (such as lysosomal storage disorders (LSD)) and as well as aggregate/plaque based diseases. Specifically, trehalose induces TFEB to not only increase the number of lysosomes per cell but also produce more active lysosomes. This is again is specifically beneficially for diseases involving compromised lysosome activity, such as lysosomal storage disorders. The combination of trehalose and/or sucrose being present in the buffer and the EVs being genetically modified to comprise a therapeutic protein, especially a therapeutic transmembrane protein are found to be particularly synergistic. Thus, in a preferred embodiment, the present invention relates to compositions comprising EVs genetically engineered to comprise an LSD enzyme or transporter or other LSD associated protein of interest and the storage buffer herein, wherein the component comprising a saccharide moiety is trehalose or sucrose. LSD enzymes, transporters and proteins of interest include for instance alpha-D-mannosidase, N-aspartyl-beta-glucosaminidase, lysosomal acid lipase, cystinosin, lysosomal associated membrane protein-2 (LAMP2), alpha-galactosidase A, acid ceramidase, alpha-fucosidase, cathepsin A, acid beta-glucosidase, beta-galactosidase, beta-hexosaminidase A, beta-hexosaminidase B, GlcNAc-1-phosphotransferase, beta-galactosylceramidase, lysosomal acid lipase, arylsulfatase A, alpha-L-iduronidase, iduronate-2-sulphatase, paran sulphamidase, acetyl alpha-glucosaminidase, acetyl CoA: alpha-glucosaminide-N-acetyltransferase, N-acetyl glucosamine-6-sulfatase, N-acetyl galactosamine-6-sulfatase, hyaluronidase, acetyl galactosamine-4-sulphatase, beta-glucuronidase, alpha-N-acetyl neuraminidase, N-actiylglucosamine-1-phosphotransferase, mucolipin-1, formylglycine-generating enzyme, palmitoyl-protein thioesterase-1, tripeptidyl peptidase I, cysteine string protein, CLN3p, CLN5p, CLN6p, CLN7p, CLN8p, acid sphingomyelinase, NPC 1, NPC 2, acid alpha-glucosidase, cathepsin K, sialin, alpha-N-acetylgalactosaminidase, GM2 activator, lysosomal acid lipase, and derivatives, regions, domains and/any combinations thereof. Importantly, in preferred embodiments, the genetically engineered EVs are exosomes engineered to comprise at least one but preferably several copies of NPC1, GBA, cystinosin, CLN3p, CLN5p, CLN6p, CLN7p, CLN8p, acid sphingomyelinase, NPC 2, acid alpha-glucosidase, cathepsin K, sialin, alpha-N-acetylgalactosaminidase, GM2 activator, etc. per EV.

The pharmaceutical formulations as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EV population as such, the drug cargo engineered into the EVs and its molecular target as well as target organ. For many applications, subcutaneous and intravenous administration are preferred and the pharmaceutical compositions as per the present invention are highly suitable for these but also other routes of administration.

Importantly, the inventors behind the present invention has optimized the components and ingredients of the compositions described herein to be compatible with human clinical use, through the use of components generally regarded as safe (GRAS) and components already being used clinically. The pharmaceutical compositions described herein may comprise various pharmaceutically acceptable carriers and/or excipients, for instance saline solutions, sugar solutions, Ringer's lactate, Ringer's acetate, Plasmalyte A, Albunorm®, heparinized solutions, carriers for lyophilization, carrier for nebulizations, as well as various other carriers and/or excipients, and naturally any combinations thereof.

In a further aspect, the present invention relates to a method for preparing the pharmaceutical compositions herein. Such methods typically comprise a one-step mixing of an EV composition comprising genetically engineered EVs carrying at least one drug cargo, with a pharmaceutically acceptable carrier, as above-mentioned. The present invention also pertains to methods and processes for producing the EV compositions as described herein. Such processes may comprise the steps of (i) culturing of EV-producing cells in a suitable culture vessel, wherein the EV-producing cells are genetically engineered to produce drug loaded, genetically modified EVs, (ii) purify and/or isolate (partly or completely, depending on the level of desired purity of the final EV composition) the EVs obtainable from the EV-producing cells, and (iii) at any point prior to, during, and/or after step (ii) mixing the EVs with the storage buffer herein. The at least one purification technique may be selected from the group comprising centrifugation, ultracentrifugation, filtration, ultrafiltration, tangential flow filtration, liquid chromatography, size exclusion liquid chromatography, ion exchange liquid chromatography, and/or any combination thereof. In a preferred embodiment, purification (also termed isolation) is carried out using a combination of filtrations, preferably tangential flow filtration, and liquid chromatography. Furthermore, this process may also comprise mixing the EV-containing storage buffer of step (iii) with a pharmaceutically acceptable carrier, as outlined above.

In yet another aspect, the present invention relates to a method for stably storing EVs, comprising the steps of (i) introducing genetically engineered EVs into the storage buffers as described herein, and (ii) storing the EV-containing storage buffer at a suitable temperature. Suitable temperatures for storage include sub-zero temperatures, although temperatures above 0° C. are also contemplated and work well for short- and medium-term storage of EVs with maintained activity, especially if the temperature is below 8° C., preferably below 5° C. For long-term storage, it is most preferable to store the EV-containing compositions at sub-zero temperatures, such as below −15° C. (preferably around −20° C.), even more preferably below −50° C. (preferably around −60° C. or −80° C.).

It shall be understood that the above described exemplifying aspects, embodiments, alternatives, and variants can be modified without departing from the scope of the invention. The invention will now be further exemplified with the enclosed examples, which naturally also can be modified considerably without departing from the scope and the gist of the invention.

### EXAMPLES

### Experiment 1. Evaluation of Engineered EV Stability

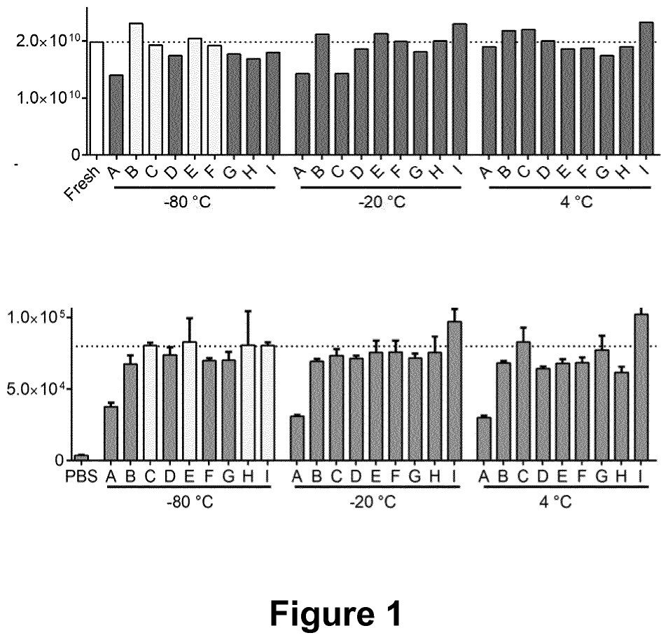

Genetically engineered and immortalized mesenchymal stromal cells (MSCs) were cultured in bioreactors. Conditioned media (CM) containing the EVs was harvested from the bioreactors. To isolate the EVs the CM was centrifuged, first at 500×g for 5 minutes to remove cells, followed by 2,000×g for 10 minutes to remove cell debris and thereafter filtrated through an 0.22 μm filter to remove any larger particles. The filtered CM was then run through a hollow fiber filter using a tangential flow filtration (TFF) system and concentrated down after diafiltration. The pre-concentrated CM was subsequently run through onto BE-SEC columns connected to a chromatography system and concentrated a cut-off spin-filter. The EVs were then stored in different buffer conditions (A-I, see table 1 below) at different temperatures (−80° C., −20° C., 4° C.). After 26 weeks storage, the EVs were thawed and the concentration of the EVs were assessed by nanoparticle tracking analysis (NTA) and compared to the NTA determined initial concentration. As shown in FIG. 1 i) the addition of albumin (recombinant human serum albumin (HSA)) was well as HEPES and/or trehalose/sucrose and/or DMSO to PBS results in unaffected concentration of EVs over time as compared to PBS only, which reduces the concentration of EVs.

### Example 2. Evaluation of Engineered EV Protein Stability During Storage

HEK293T cells stably engineered to express a GFP chimeric peptide fused to the common EV sorting protein CD63 (CD63-GFP, resulting in GFP display inside the modified EVs) were cultured in bioreactors. Conditioned media (CM) containing the EVs was harvested from the bioreactors. To isolate the EVs the engineered CM was centrifuged, first at 500×g for 5 minutes to remove cells, followed by 2,000×g for 10 minutes to remove cell debris and thereafter filtrated through an 0.22 μm filter to remove any larger particles. The filtered CM was then run through a hollow fiber filter using a tangential flow filtration (TFF) system and concentrated down after diafiltration with PBS. The pre-concentrated CM was subsequently run through onto BE-SEC columns connected to a chromatography system and concentrated using cut-off spin-filter. The GFP-positive EVs were then stored in different buffer conditions (A-I, see table 1 below) at different temperatures (−80° C., −20° C., 4° C.). After 26 weeks storage, the EVs were thawed and the EV protein stability was assessed by the presence of GFP, which was detected using a spectrometer (SpectraMax). As shown in FIG. 1 ii) EVs stored in PBS (or NaCl) with the addition of HSA and/or HEPES and trehalose have a higher signal of GFP compared to EVs stored in PBS only at all temperatures measured, demonstrating that these additives increase the stability of EVs. Similar results were obtained using another engineered strategy, whereby GFP was fused to the extravesicular part of the exosome protein Lamp2B. In parallel experiments, sucrose, lactose, maltose, mannitol, sorbitol, were also evaluated and showed similar stability-enhancing effects (data not shown).

### Example 3: Cellular Uptake of EVs as Readout for Retained EV Functionality

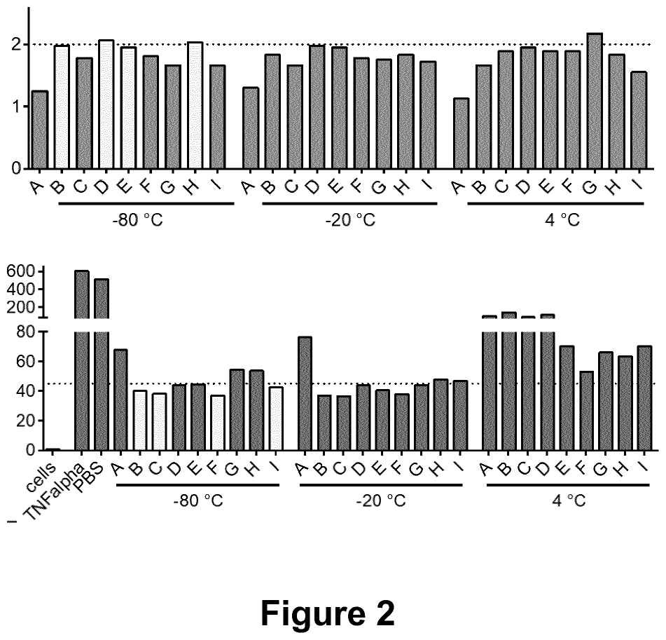

HEK293T cells stably expressing a GFP chimeric peptide fused to the common EV sorting proteins CD63 or Lamp2B (CD63-GFP, resulting in GFP display inside EVs, and Lamp2b-GFP, resulting in GFP display on the outside of EVs) were cultured in bioreactors. Conditioned media (CM) containing the EVs was harvested from the bioreactors. To isolate the EVs the CM was centrifuged, first at 500×g for 5 minutes to remove cells, followed by 2,000×g for 10 minutes to remove cell debris and thereafter filtrated through an 0.22 μm filter to remove any larger particles. The filtered CM was then run through a 300 Kd hollow fiber filter using a tangential flow filtration (TFF) system and concentrated down to approx. 40-50 mL after diafiltration with PBS. The pre-concentrated CM was subsequently run through onto BE-SEC columns connected to a chromatography system and concentrated using a 10 kDa molecular weight cut-off spin-filter. The GFP-positive EVs were then stored in different buffer conditions (A-I, see table 1 below) at different temperatures (−80° C., −20° C., 4° C.). After 26 weeks storage, the EVs were thawed and incubated with Huh7 cells. After 6 hours incubation the cellular uptake in Huh7 cells of the GFP-positive EVs was assessed by flow cytometry. As shown in FIG. 2 i) EVs stored in PBS (or NaCl) with the addition of HAS and HEPES, and/or trehalose has a higher cellular uptake, as measured by GFP signal, compared to EVs stored in PBS only at all temperatures measured, demonstrating that these additives increase the stability of EVs. In parallel experiments, HEPES was replaced by BES, DIPSO, TES, acetamidoglycine, HEPPSO, EPS, HEPPS, tris, glycinamide, glycylglycine, and AMPSO, combined with various other sugars such, which resulted in similar results (data not shown).

### Example 4: Anti-Inflammatory Activity as Readout for Preserved EV Functionality

Immortalized mesenchymal stromal cells (MSCs) genetically engineered to stably express TNFalpha decoy receptors in a chimeric protein with an EV sorting domain fusion (TNFR fused to CD63, for display on the extravesicular surface of the membrane of the EVs) were cultured in bioreactors. Conditioned media (CM) containing the EVs was harvested from the bioreactors. To isolate the EVs the CM was centrifuged, first at 500×g for 5 minutes to remove cells, followed by 2,000×g for 10 minutes to remove cell debris and thereafter filtrated through an 0.22 μm filter to remove any larger particles. The filtered CM was then run through a 300 Kd hollow fiber filter using a tangential flow filtration (TFF) system and concentrated down to approx. 40-50 mL after diafiltration with PBS. The pre-concentrated CM was subsequently run through onto BE-SEC columns connected to a chromatography system and concentrated using a 10 kDa molecular weight cut-off spin-filter. The EVs were then stored in different buffer conditions (A-I, see table 1 below) at different temperatures (−80° C., −20° C., 4° C.). NF-κB reporter (Luc)-HEK293 cells expressing the NF-κB-luciferase reporter gene, was employed to assesses the anti-inflammatory capacity of the MSC derived EVs displaying TNFalpha decoying receptors. The level of inflammation was assessed by luciferase activity in the unstimulated cells (cells), TNFalpha stimulated cells (TNFalpha) and TNFalpha stimulated cells with PBS (PBS) or EVs stored in the different buffers (A-I) and different temperatures (−80° C., −20° C., 4° C.) for 26 weeks. As shown in FIG. 2 ii) EVs stored in PBS with the addition of HSA and/or HEPES and/or trehalose and/or DMSO display a retained anti-inflammatory capacity, as measured by decreased luciferase levels, compared to EVs stored in PBS, despite the presence of substantial amounts of HAS which was assumed to block the interaction between the receptor and its ligand as a result of corona formation. Furthermore, to evaluate pharmaceutically acceptable carriers, EVs from this experiment were subsequently formulated pharmaceutically acceptable carriers, for instance saline solution, a 5% glucose solution, Ringer's acetate, and Tyrode's solution, which all proved to be highly functional carriers for in vivo experiments in a TNBS-induced colitis model, where the TNFalpha decoy function of the EVs were evaluated (data not showed).

TABLE 1
Examples of buffers.

| Buffer | | Additive | Additive | Additive | Additive |
|---|---|---|---|---|---|
| reference | Buffer | 1 | 2 | 3 | 4 |
| A | PBS | | | | |
| B | PBS | HSA | HEPES or tris | | |
| C | PBS | HSA | | | |
| D | PBS | HSA | trehalose or | | |
| | | | sucrose | | |
| E | PBS | HSA | HEPES or | trehalose | PEG |
| | | | acetamido- | or | |
| | | | glycine | sucrose | |
| F | PBS | HSA | HEPES | Trehalose | DMSO |
| G | NaCl | HSA | HEPES or, | | |
| | | | HEPPS | | |
| H | NaCl | HSA | HEPES or | Trehalose | |
| | | | glycylglycine | | |
| I | ExoCap | | | | |

1. A storage buffer for genetically modified extracellular vesicles (EVs), comprising:
(i) an aqueous solution comprising at least one buffering agent;
(ii) at least one component comprising a saccharide moiety; and,
(iii) at least one exogenously added polypeptide.

2. The storage buffer according to claim 1, wherein the EVs are genetically modified to comprise at least one drug cargo present inside the EV, in association with the EV membrane, and/or as an extravesicular drug cargo on the external side of the EV membrane.

3. The storage buffer according to claim 2, wherein the drug cargo is a transmembrane polypeptide drug.

4. The storage buffer according to claim 1, wherein the aqueous solution is an isotonic solution.

5. The storage buffer according to claim 1, wherein the aqueous solution is phosphate-buffered saline (PBS), Dulbecco's PBS, Hank's buffered salt solution (HBSS), Earle's balanced salt solution (EBSS), Modified Eagle's Medium, Minimum Essential Medium, Dulbecco's Modified Eagle's Medium (DMEM), Glasgow Minimum Essential Medium (GMEM), RPMI1640, IMDM, Ham F10, Ham's F12, or any combination thereof.

6. The storage buffer according to claim 1, wherein the at least one buffering agent is MES, bis-tris methane, ADA, ACES, bis-tri propane, PIPES, MOPSO, cholamine chloride, MOPS, BES, TES, HEPES, DIPSO, MOBS, acetamidoglycine, TAPSO, TEA, POPSO, HEPPSO, EPS, HEPPS, tricine, tris, glycinamide, glycylglycine, HEPBS, bicine, TAPS, AMPB, CHES, AMP, AMPSO, CAPSO, CAPS, CABS, or any combination thereof.

7. The storage buffer according to claim 1, wherein the at least one component comprising a saccharide moiety comprises a monosaccharide, a disaccharide, a trisaccharide, an oligosaccharide, a polysaccharide, a sugar alcohol, or any combination thereof.

8. The storage buffer according to claim 1, wherein the at least one exogenously added polypeptide is serum albumin, human serum albumin, recombinant serum albumin, recombinant human serum albumin, Albunorm®, individual amino acids, and/or any combinations thereof.

9. The storage buffer according to claim 1, further comprising an excipient, wherein the excipient is selected from a cryoprotectant, a lyophilization excipient, a preservative, an antibiotic, an organic solvent, an inorganic salt (ammonium nitrate, calcium chloride, potassium hydrate), a carbohydrate, an amino acid, a vitamin, a fatty acid, a serum component, a trace element, or any combination thereof

10. The storage buffer according to claim 9, wherein the excipient is PEG (polyethylenglycol), heparin, propylene glycol, ethylene glycole, EDTA, glycerol, dithiothreitol (DTT), co-block polymers, or any combination thereof.

11. The storage buffer according to claim 1, wherein the EV comprises at least one drug cargo.

12. The storage buffer according to claim 11, wherein the at least one drug cargo is a nucleic acid molecule, a protein, a peptide, a small molecule, and/or any combination thereof.

13. An EV composition, comprising:
(i) an aqueous solution comprising at least one buffering agent;
(ii) at least one component comprising a saccharide moiety;
(iii) at least one exogenously added polypeptide; and,
(iv) at least one population of genetically engineered EVs.

14. The EV composition according to claim 13, wherein the EVs are genetically modified to comprise at least one drug cargo present inside the EV, in association with the EV membrane, and/or as an extravesicular drug cargo on the external side of the EV membrane.

15. The EV composition according to claim 14, wherein the drug cargo is a transmembrane polypeptide drug.

16. The EV composition according to claim 11, wherein the EVs constituting the EV population comprises at least one drug cargo.

17. A pharmaceutical composition comprising the EV composition according to claim 13 and a pharmaceutically acceptable carrier.

18. (canceled)

19. The pharmaceutical composition according to claim 17, for use in the treatment, prophylaxis, and/or alleviation of a disease, disorder, and/or condition selected such as cancer, autoimmune disease, inflammatory disease, cardiovascular disease, neurodegenerative disease, neuroinflammatory disease, neuromuscular disease, lysosomal storage disease, neurometabolic disease, inherited liver disease, and any other disease.

20. The pharmaceutical composition according to claim 17, wherein the pharmaceutically acceptable carrier is saline solution, a sugar-containing solution, Ringer's lactate, Tyrode's solution, Ringer's acetate, Plasmalyte A, Albunorm®, a heparinized solution, a carrier for lyophilization, a carrier for nebulization, and/or any combination thereof.

21. A method for preparing the pharmaceutical composition according to claim 17, comprising the step of mixing the EV composition comprising:
(i) an aqueous solution comprising at least one buffering agent;
(ii) at least one component comprising a saccharide moiety;
(iii) at least one exogenously added polypeptide; and,
(v) at least one population of genetically engineered EVs
with a pharmaceutically acceptable carrier.

22. The method according to claim 21, wherein the pharmaceutically acceptable carrier is saline solution, a sugar-containing solution (such as 5% glucose solution), Ringer's lactate, Ringer's acetate, Tyrode's solution, a heparinized solution, a carrier for lyophilization, a carrier for nebulizer, or any combination thereof.

23. A process for producing an EV composition according to claim 13, comprising the steps of:
(i) culture EV-producing cells;
(ii) purify EVs obtainable from the EV-producing cells using at least one purification technique selected from the group comprising centrifugation, ultracentrifugation, filtration, ultrafiltration, tangential flow filtration, liquid chromatography, size exclusion liquid chromatography, ion exchange liquid chromatography, and/or any combination thereof;
(iii) at any point prior to, during, and/or after step (ii) mixing the EVs with the storage buffer according to claim 1.

24. The process according to claim 23, further comprising mixing the EV-containing storage buffer of step (iii) with a pharmaceutically acceptable carrier.

25. A method for stably storing EVs, comprising the steps of:
(i) introducing EVs into the storage buffer according to claim 1; and
(ii) storing the EV-containing storage buffer at a suitable temperature.

26. The method according to claim 25, wherein a suitable temperature is below 8° C., preferably below 4° C., more preferably below −15° C., even more preferably below −50° C. such as −80° C.
